SEQUENCE LISTING (2 sequences)

SEQ ID NO: 1 - DNA (genomic DNA), 24 bp; both-stranded, linear
atcatcgacggcaacctcggagac

SEQ ID NO: 2 - DNA (genomic DNA), 25 bp; both-stranded, linear
caccattcccaagctaaaccagtgc

(19) United States
(12) Patent Application Publication
(10) Pub. No.: US 2004/0053213 A1
(43) Pub. Date: Mar. 18, 2004

(54) Method for detecting microorganisms using PCR amplicons and microsphere agglutination

(75) Inventors: Clarence I. Kado, Davis, CA (US); Shaw-Jye Wu, Jung-Li City (TW); Alexander Chan, Southbury, CT (US)

Correspondence Address: Paul D. Greeley, Esq., Ohlandt, Greeley, Ruggiero & Perle, L.L.P., 10th Floor, One Landmark Square, Stamford, CT, 06901-2682, US

(73) Assignee: Avenir Genetics LLC

(21) Appl. No.: 10/232,439

(22) Filed: Aug. 29, 2002

(51) Int. Cl. C12Q 1/70; C12Q 1/68

(52) U.S. Cl. 435/5; 435/6

(57) ABSTRACT

The present invention discloses a rapid and easily performed method for the determination of the presence or absence of bacteria in a sample. The method involves using polymerase chain reaction nucleic acid primers to amplify target microbial sequences and thereafter detecting the presence or absence of amplicons using microsphere agglutination.

### BACKGROUND OF THE INVENTION

1. Field of the Invention

The present invention relates to the field of microbiology and particularly to the identification of a specific microbial nucleotide sequence as a diagnostic method for the determination of the presence or absence of a particular microorganism.

2. Description of the Prior Art

The development of polymerase chain reaction (PCR) technology has enabled the detection of small amounts of nucleic acid material. It has proven to be one of the most popular and valuable techniques used for the detection of microbial contaminants in foods and a variety of other biological, environmental and forensic samples. The high specificity and amplification capability of PCR technology has shortened and often eliminated the lengthy enrichment and isolation procedures that are prerequisites for conventional biochemical and immunological-based microbial detection methodologies. In view of its high sensitivity, discrimination capability, and rapid reaction time, PCR is certainly one of the most important techniques employed in microbial detection and identification.

However, in spite of its demonstrated efficacy and value, there are several limiting factors that remain to be improved before the potential of PCR technology in microbial detection and identification can be fully exploited. For example, all PCR-based methods are subjected to amplicon detection. The detection of amplified DNA is usually accomplished by agarose gel electrophoresis and subsequent viewing of the ethidium bromide-stained PCR product. This timely and cumbersome approach has remained largely unchanged since the advent of PCR. Although recent approaches using fluorescence resonance energy transfer (FRET) technology has provided an alternative for faster amplicon detection [see Fitzgerald, D. A., The Scientist. 14, 31 (2000)], the prerequisite of special probes and expensive photo optical equipment, however, compromised its general usability. Hence, in the present invention, we disclose a novel, rapid yet inexpensive method of detecting PCR amplicons. In a preferred embodiment using biotin-labeled primers, the presence of the PCR amplified DNA fragments could be detected by streptavidin-coated microsphere agglutination. PCR was carried out using a regular thermal cycler. No special equipment was required for the agglutination assay, and the results were determined within two minutes.

Microsphere agglutination has been widely applied to detect or monitor infectious microorganisms like Helicobacter pylori (Midold et al., 2001), S. aureus [see van Griethuysen et al., J. Clin. Microbiol. 39, 86-89 (2001)] Salmonella enterica [see Veling J. et al., Microbiol. 38:4402-4407 (2000)], E. coli [see Huang Y. H. et al., European J. Clin. Microbiol. Infectious Dis. 20, 97-103 (2001)], Brucella spp. [see Orduña, A. et al., J. Clin. Microbiol. 38, 4000-4500 (2000)] as well as many other bacteria, protozoa, and even viruses [see March, J. B., et al., J. Clin. Microbiol. 38, 4152-4159 (2000); Al-Yousif, Y., et al., Clin. Diag. Lab. Immunol. 8, 496-498 (2001); Lindsay D. S., etal., Vet. Parasitol. 95,179-186 (2001)]. The same technique is also useful for other studies and assays such as blood typing, autoimmune disease diagnosis [see Alaedini, A., et al., J. Clin Lab. Anal. 15, 96-99 (2001)], safety control for live viral vaccines [see Zhang, D. L., et al., Cell Biol. Intnl. 25, 997-1002 (2001)], microbial antibiotic resistance assay [see Khatib Jafri, A., et al., Diag. Microbiol. Infectious Dis. 36, 57-59 (2000)], interactive mechanisms between parasitic microorganisms and their hosts [see Blandorf, D. C. M., et al., Appl. Environ. Microbiol. 60, 1726-1733 (1994); Benyagoub, M. et al., Mycobiol. Res. 100, 79-86 (1996); Inbar, J. et al., Critical Rev. Biotechnol. 17, 1-20 (1997)], etc. However, most of these applications rely on the presence and the quantity of microbial antigens and the availability of specific antibodies against them. The present invention is the first report of an agglutination test that detects PCR amplicons. The combination of the easy implementation of microsphere agglutination and the high sensitivity and specificity of PCR, will not only benefit food safety and clinical microbial diagnosis, but will also facilitate many other applications.

Despite its efficacy and value being demonstrated, several limiting factors intrinsic to PCR, however, remain to be improved before its potential in microbe detection and identification can be fully exploited. For example, a compressor-operating thermal cycler, whose size, weight, and power consumption restricts its portability, can significantly impair the usefulness of a PCR-based pathogen detection method in field applications. This is why there have been great efforts in incorporating solid-state, thermoelectric (Peltier-effect) cooling systems for smaller, lighter battery-powered thermal cyclers [see S. Beck, The Scientist 11:24 (1997) “Heat wave: the thermal cycles of 1997”; MiniCycler™ M J Research, Inc., Boston, Mass.; Smart Cycler,Cepheid, Inc., Sunnyvale Calif.; Meisenholder, G., The Scientist 13, 17 (1999)]. On the other hand, all PCR-based detection methods are subjected to amplicon-revealing procedures. Conventionally, a post-PCR agarose gel electrophoresis is employed followed by fluorescent dye staining for visual examination. This relatively time-consuming and labor-intensive approach is gradually being replaced by an array of real-time PCR techniques [see Orlando, C., eta/., Clin. Chem. Lab. Med. 36, 255-269 (1998)]. Although these techniques have proven to be fast and capable of high throughput, the need of designing sequence specific fluorescent probes and the requirement of dedicated, expensive photo optical devices, however, diminish their field worthiness and restrict their prevalence in clinical laboratories.

Very simple, fast, easy to perform, and cost effective methods for amplicon detection that are field worthy and can be widely adapted have not yet been developed. In view of this goal, we have designed and developed a novel method using microsphere agglutination and kit related thereto. Microsphere agglutination technique is based on the bridge molecules to bring microspheres together from their suspension status and to form visible reticulated clots. Such an approach has been broadly applied in immunological-based analysis but has never been applied to amplicon detection. By using biotin labeled forward and reverse primers concurrently, we demonstrated that target PCR amplicon, whose both ends are then labeled with biotins, agglutinate streptavidin coated microspheres. This makes use of the high affinity reaction between streptavidin and biotin for general immobilization of biotinylated compounds. No other probes and post-PCR modifications were required for the assay and the result could be visualized within two minutes. The simplicity and portability suggest its worthiness in field applications, and the efficiency and economization indicate its great value in routine microbe monitoring in food industry and other bio-hazardous laboratories.

Microbeads are used in agglutination assays of activated platelets in blood. For example, Accumetrics, San Diego, Calif. markets the product Ultegra Rapid Platelet Function Assay. Activated platelets bind and agglutinate fibrinogen-coated beads. The agglutination reaction is quantified by the degree of light transmittance. For our microsphere system, as long as the amplicons can bind microspheres, the agglutination reaction will take place. The biotin-avidin or streptavidin affinity reaction is one of the strongest (10⁻²⁸ M) known in biology.

Accordingly, it is an object of the invention to provide a method for the rapid detection of polymerase chain reaction amplicons.

It is another object of the invention to provide a method for determination of polymerase chain reaction amplicons that can be easily visualized.

It is yet another object of the invention to provide a method for rapid determination of polymerase chain reaction amplicons of microorganisms that can be identified visually.

These and other objects and advantages of the present invention and equivalents thereof are achieved by methods for the detection of polymerase chain reaction amplicons§ from bacteria using microsphere agglutination.

### SUMMARY OF THE INVENTION

For rapid and inexpensive detection of PCR amplicons, a novel microsphere agglutination assay was developed. PCR was carried out using biotin labeled forward and reverse primers and the amplified DNA fragments were able to agglutinate streptavidin-coated microspheres (5.7 μm in diameter). The size range of microsphere can be larger than 5.7 (approximately the size of red blood cells) as long as the agglutination reaction can take place. No post-PCR purification of amplicon was needed when initial primer concentrations were at 250 nM. Agglutination results could be identified visually within two minutes without any additional equipment or device. In one embodiment using listeriolysin gene (lisA) specific biotinylated primers, Listeria monocytogenes lisA⁺ cells were detected and identified in a sample among Salmonella typhimurium, Staphylococcus aureus, Campylobacter jejuni and E. coli O157:H7. The simplicity of the present invention offers considerable savings of time and cost, is useful for various studies and field applications, and may detect the presence or absence of a target microorganism in a sample.

The present invention discloses a method for detecting the presence or absence of a target microorganism in a sample comprising: contacting DNA from a target microorganism with at least one primer capable of hybridizing to a portion of the DNA; amplifying the DNA using polymerase chain reaction; and detecting the presence or absence of said amplified DNA by microsphere agglutination. Microsphere agglutination may optionally be promoted with ultrasound. Also, microsphere agglutination may optionally be measured quantitatively, preferably spectrophotometrically including but not limited to light scattering or UV/Vis spectrophotometry. The target microorganism may be any microorganism containing nucleic acid, including bacteria, fungi, viruses, or protozoans. A preferred embodiment is the detection of the presence or absence of a pathogenic bacterium, including but not limited to Listeria, Escherichia, Salmonella, Campylobacter, Staphylococcus, and Streptococcus. Other potential targets are species of Legionella, Bartonella, Bordetella, Brucella, Burkholderia, Klebsiella, Citrobacter, Yersinia, Shigella, Morganella, Pseudomonas, and Bacillus anthracis. Any convenient primer effective in hybridizing and amplifying DNA by PCR from a selected target microorganism may be employed. Design and construction of such primers is well known in the art. A preferred embodiment is a biotinylated primer agglutinating streptavin-coated microspheres. A preferred primer pair for the detection of Listeria monocytogenes is 5′-biotin-ATC ATC GAC GGC MC CTC GGA GAC-3′ and 5′-biotin-CAC CAT TCC CM GCT AAA CCA GTG C-3′.

The present invention also discloses a kit for use in the polymerase chain reaction detection of the presence or absence of a target microorganism comprising: a pair or biotinylated amplification primers capable of hybridizing with the DNA of a target microorganism; a polymerase, reagents and buffers necessary to effect DNA amplification; and microspheres capable of agglutinating in the presence of biotinylated amplified DNA of said target microorganism.

### BRIEF DESCRIPTION OF THE DRAWINGS

FIG. 1 is a comparison of biotin-labeled PCR amplicons agglutinating streptavidin-coated microspheres (+) and TRIS-EDTA buffer (−). PCRs were carried out using biotinylated; lisA⁺ specific primers. Amplicons were purified and mixed with streptavidin-coated microspheres. (−) Using Tris-EDTA buffer as template during PCR; (+) using cell lysate from L. monocytogenes lisA⁺ strain as template during PCR.

FIG. 2 is a Agarose gel (2.0%) electrophoretogram of PCR amplified products from cell lysate of L. monocytogenes lisA⁺ using various concentrations of biotinylated lisA⁺ specific primers. Each lane was loaded with 10 μl of solution of the end PCR product. Lane M, 100 bp DNA ladder. Lane 1, negative control using Tris-EDTA buffer as template, initial concentrations for both forward and reverse primers were 500 nM. Lanes 2-6, initial primer concentrations were 1000, 500, 250, 100 and 50 nM respectively.

FIG. 3 is an agglutination assay of raw PCR solutions derived from those of FIG. 2. Two micro liters of PCR end solutions were mixed with 3 ml of streptavidin-coated microspheres. Dot 1, negative control; Dots 2-6, amplicons derived from PCR using 1000, 500, 250, 100 and 50 nM of primers respectively.

FIG. 4. Agarose gel (2.0%) electrophoretogram of PCR amplified products from cell lysates of S. typhimurium (lane 2), L. monocytogenes (lane 3), S. aureus (lane 4), C. jejuni (lane 5) and E. coli O157:H7 (lane 6) using biotinylated lisA⁺ specific primers whose initial concentration was 250 nM each. Negative control was carried out using Tris-EDTA instead of cell lysate as template. Each lane was loaded with ten μl of end PCR solution.

FIG. 5 is an agglutination assay of raw PCR solutions derived from those described in FIG. 4. Two micro liters of PCR end solutions were mixed with 3 ml of streptavidin-coated microspheres. Dot 1, negative control; Dots 2-6, end PCR solutions from S. typhimurium, L. monocytogenes, S. aureus, C. jejuni and E. coli O157:H7 respectively.

### DETAILED DESCRIPTION OF THE INVENTION

In the present invention, an amplification primer is an oligonucleotide primer for amplification of a target nucleic acid sequence by extension of the primer after hybridization to the target sequence. Amplification primers are generally about 10-75 nucleotides in length, and preferably about 15-50 nucleotides in length. The target nucleotide binding sequence confers hybridization specificity on the amplification primer. The target binding sequence is the portion of the primer which determines its target specificity. The amplification primer may consist of target binding sequence or may have target binding sequence and additional modification. For example, amplification of a target sequence according to the present invention uses biotinylated primer in the Polymerase Chair Reaction (PCR). It is understood that any number of amplification primers suitable for hybridizing with target microbial DNA and PCR may be employed in the present invention for detecting the presence or absence of a target microorganism.

As used herein, the term target or target sequence refers to nucleic acid sequences to be amplified that are derived from a microorganism. These include the original nucleic acid sequence to be amplified, the complementary second strand of the original nucleic acid sequence to be amplified and either strand of a copy of the original sequence which is produced by the amplification process. These copies serve as amplifiable targets by virtue of the fact that they contain copies of the sequence to which the amplification primers hybridize. Copies of the target DNA sequence that are generated during the amplification reaction are referred to as amplification products or amplicons. Amplicon refers to the product of the amplification reaction generated through the extension of either or both of a pair of amplification primers. An amplicon may contain exponentially amplified nucleic acids if both primers utilized hybridize to a target sequence. Alternatively, amplicons may be generated by linear amplification if one of the primers utilized does not hybridize to the target sequence. Thus, this term is used generically herein and does not necessarily imply the presence of exponentially amplified nucleic acids.

Bacterial strains. The following bacterial strains were used in the present invention: enterohemorrhagic E. coli O157:H7 slt⁺ strain containing Shiga-like toxin gene (GenBank accession no. AB048837); Salmonella typhimurium stn⁺ strain containing an enterotoxin gene (GenBank accession no. L16014); Campylobacter jejuni cdtB⁺ strain containing the cytolethal distending toxin gene (GenBank accession no. AF038283); Listeria monosytogenes lisA⁺ strain containing listeriolysin gene (GenBank accession no. X15127); and Staphylococcus aureus entA⁺ strain containing enterotoxin A gene (GenBank accession no. Ml 8970).

Culture media. Media used for growing bacterial cells were LB agar for E. coli [Miller], Brucella agar (Difco 0964) for C. jejuni, brain heart infusion agar (Difco 0418) for L. monocytogenes and nutrient agar (Difco 0001) for Salmonella typhimurium, and Staphylococcus aureus. All growing temperatures were at 37° C., and incubation for C. jejuni was in a microaerobic environment.

PCR primers and amplification condition. The gene encoding L. monocytogenes listerin gene (GenBank accession no. X15127) was chosen for PCR and microsphere agglutination assay. The primer pairs for PCR were 5′-biotin-ATC ATC GAC GGC MC CTC GGA GAC-3′ and 5′-biotin-CAC CAT TCC CM GCT AAA CCA GTG C-3′. The size of the expected PCR amplicon is 404 bp.

DNA template was isolated by transferring a single, isolated colony from an agar plate to 200 μl of a solution consisting of 0.5% Triton X-100, 20 mM Tris (pH 8.0), 2 mM EDTA and boiled for 10 minutes to lyse the cells (Fratamico et al., 1995). For positive control, 5 μl of the colony lysate, 5 μl of 10 μM primer each, 8 μl 25 mM MgCl₂, 2 μl of 10 mM dNTP each, 10 μl of GeneAmp AmpliTaq Gold 10× buffer and 1 μl of 5 units/μl AmpliTaq Gold DNA polymerase (Applied Biosystem, Foster City, Calif.) were added to 58 μl of ddH₂O to make up a 100 μl reaction volume. Amplification was carried out in a Perkin-Elmer Gene Amp 2400 thermal cycler. An initial denaturation of 94° C. for 10 minutes was followed by 40 cycles of denaturation at 94° C. for 30 sec and annealing/polymerization at 68° C. for 90 sec. After the cycles, a final extension period was set at 72° C. for 5 minutes. Following amplification, 10 μl of the PCR reaction was analyzed by agarose (2.0%) gel electrophoresis and subsequent visualization with ethidium bromide staining.

Microsphere agglutination by PCR amplicons. For testing the ability of PCR product to agglutinate microspheres, amplicons were purified from PCR reagents and excess primers by using Qiaquick PCR purification kit (QIAGEN Inc., Valencia, Calif.) with conditions according to manufacturer's instruction manual. Two μl of purified amplicon were mixed with 3 μl of 10% solid, streptavidin coated, 5.7 μm diameter microspheres (Bangs Laboratories, Inc. Fishers, Ind.). The mixture was spread on a glass slide to form a round film with about 5 mm in diameter, and the slide was slightly tilted back and forth a few times to facilitate the agglutination.

PCR conditions and primer specificity. A single DNA fragment, visualized by agarose electrophoresis and ethidium bromide staining, was amplified when using the listeriolysin gene specific primers and DNA template prepared from L. monocytogenes lisA⁺ strain. The length of the DNA fragment was measured to be around 404 bp, which is the expected size of the amplicon. No amplification products were observed when the template was substituted by DNA from E. coli, S. typhimurium, C. jejuni, or S. aureus. The consistency and reproducibility of the PCR results indicated that the high-melting-point:primers and the two-step amplification cycles were sufficient for the PCR in this study.

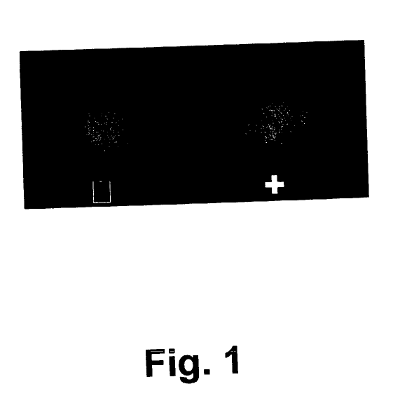

Microsphere agglutination. PCR amplicon amplified from L. monocytogenes lisA⁺ total DNA with lisA gene specific, biotin labeled primers was purified using a QIA quick PCR purification system according to the manufacturer (Cat. No. 28104; Qiagen, Inc. 28159 Stanford Avenue, Valencia, Calif. 91355). When mixed with streptavidin coated microsphere and smeared on a glass slide, the homogenous suspension look of the mixture gradually turned to sandy facade as the microspheres granulated. This manifestation occurred within two minutes after blending. A negative control using no DNA template during PCR amplification, on the other hand, did not change the appearance of the mixture at all (FIG. 1).

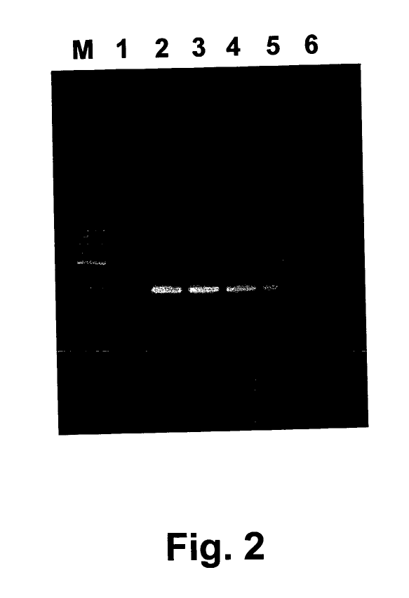

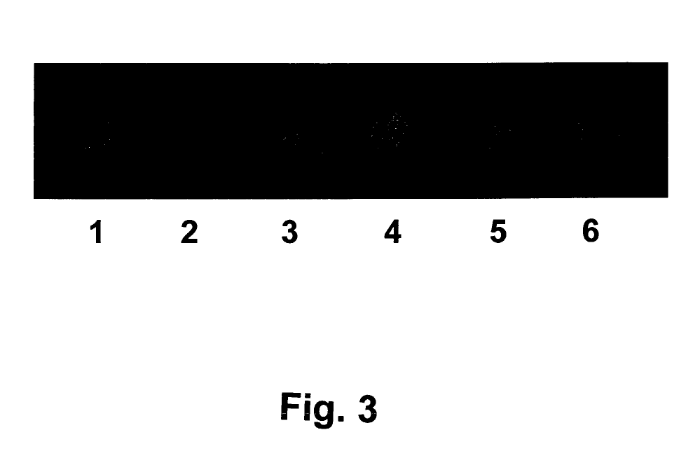

Interference of biotin labeled primers on microsphere agglutination. To measure the inhibitory effect of the remaining, free, biotin labeled primers in the agglutination reaction, PCRs with different initial primer concentrations were executed and the post-PCR solutions were applied directly to microsphere agglutination assays without any sort of amplicon purification. As shown in FIG. 2, the amounts of amplicons declined as the initial primer concentrations were sequentially reduced from 1 μM to 50 nM. Applying these post-PCR products to agglutination assay showed that visible granules appeared only when the initial primer concentrations were below 500 nM, and were the most obvious when the initial primer concentration was 250 nM (FIG. 3). The least amount of primers used in the series was 50 nM, at which concentration noticeable agglutination could still be observed.

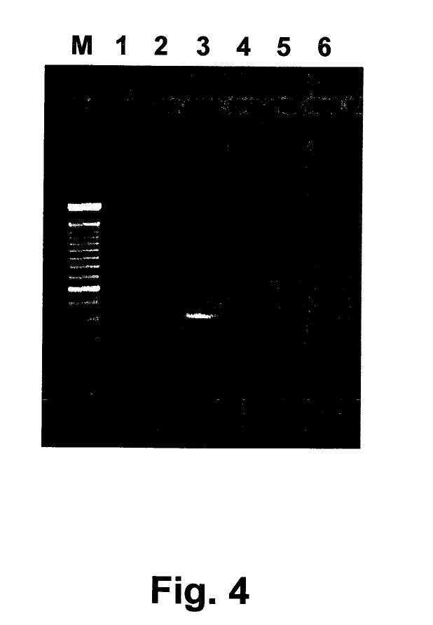

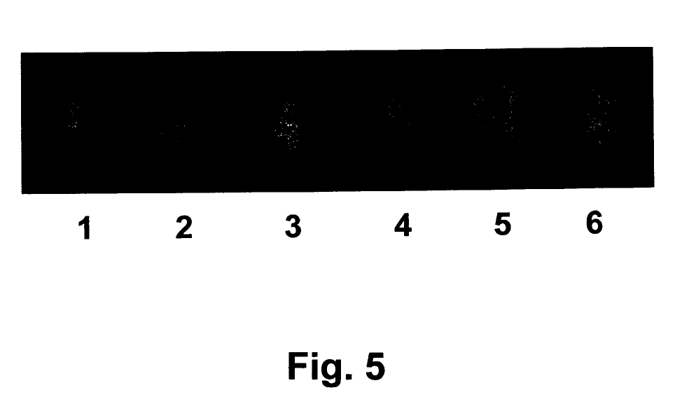

Detection and identification of microbes using amplicon mediated agglutination assay. Total DNA from S. typhimurium stn⁺, L. monocytogenes lisA⁺, S. aureus entA⁺, C. jejuni cbtB⁺ and E. coli O157:H7 slt⁺ strains were prepared respectively for PCRs with 250 nM lisA specific primers. Following amplification, 10 μl of the,PCR reactions were analyzed by agarose (2.0%) gel electrophoresis and subsequent visualization with ethidium bromide. Results showed that a DNA fragment was amplified from total DNA of L. monocytogenes lisA⁺ but not from those of other strains (FIG. 4). On the other hand, 2 μl of the PCR reactions were mixed with 3 μl of 10% solid streptavidin-coated microsphere solution and smeared on a glass slide. While the microspheres mixed with the PCR product from L. monocytogenes lisA⁺ agglutinated, those mixed with PCR from other strains suspended homogeneously and continuously (FIG. 5).

The amplicon-detecting microsphere agglutination assay embodiments of the present invention are basic in nature and may conveniently be modified within the spirit of the invention. Despite the present form relies heavily on the specificity of PCR primers to minimize false positive results, it can still function as an excluding test for quick sample screening. The optimal mass and size of microsphere for best agglutination result can be determined by those skilled in the art. Optionally, ultrasonic waves can be applied in the present invention to increase the contact between microspheres, that is to promote the formation of agglutinates and to enhance the assay sensitivity [see Ellis, R. W., et al., J. Med. Microbiol. 49, 853-859 (2000); Doubrovski, V. A., et al., Ultrasound in Med. & Biol. 26, 655-659 (2000); Sobanski, M. A., et al., J. Immunoassay 2000)]. Furthermore, laser light scattering or UV/Vis spectrophotometry can be employed to measure agglutinates quantitatively [Antony, T., et al., J. Biochem & Biophys. Methods 36, 75-85 (1998); Narayanan, S., et al., Transfusion 39, 1051-1059 (1999)]. Finally, combining all these techniques will allow mass and automatic operations possible.

Although the present invention describes in detail certain embodiments, it is understood that variations and modifications exist known to those skilled in the art that are within the invention. Accordingly, the present invention is intended to encompass all such alternatives, modifications and variations that are within the scope of the invention as set forth in the following claims.

What is claimed is:

1. A method for detecting the presence or absence of a target microorganism in a sample comprising:
contacting DNA from a target microorganism with at least one primer capable of hybridizing to a portion of said DNA;
amplifying said DNA using polymerase chain reaction;
detecting the presence or absence of said amplified DNA by microsphere agglutination.

2. The method of claim 1, wherein said microorganism is a bacterium, fungus, virus, or protozoan.

3. The method of claim 1, wherein the at least one primer is the primer pair 5′-biotin-ATC ATC GAC GGC AAC CTC GGA GAC-3′ and 5′-biotin-CAC CAT TCC CM GCT AAA CCA GTG C-3′ and the target microorganism is Listeria monocytogenes.

4. The method of claim 1, wherein said at least one primer is biotinylated.

5. The method of claim 1, further comprising promoting microsphere agglutination with ultrasound.

6. The method of claim 1, wherein said microorganism is a pathogenic bacterium.

7. The method of claim 6, wherein said bacterium is selected from the group consisting of Listeria, Escherchia, Salmonella, Campylobacter, Staphylococcus, and Streptococcus, Yersinia, Shigella, Morgenella, Citrobacter, Klebsiella of the Enterobacteriaceae family of pathogens; Bacillus anthracis, Brucella, Bartonella, Bortedella, Legionella and Burkholderia.

8. The method of claim 1, wherein said microorganism is Listeria monocytogenes.

9. The method of claim 1, wherein said microsphere agglutination comprises agglutinating streptavidin-coated microspheres.

10. The method of claim 9, wherein said streptavidin-coated microspheres are aggutinated by biotinylated amplicons.

11. The method of claim 1, further comprising measuring microsphere aggutination quantitatively.

12. The method of claim 1 1, wherein said microsphere aggutination is measured spectrophotometrically.

13. The method of claim 12, wherein said microsphere agglutination is measured by light scattering or UV/Vis spectrophotometry.

14. A kit for use in the polymerase chain reaction detection of the presence or absence of a target microorganism comprising:
a pair or biotinylated amplification primers capable of hybridizing with the DNA of a target microorganism;
a polymerase, reagents and buffers necessary to effect DNA amplification; and
microspheres capable of agglutinating in the presence of biotinylated amplified DNA of said target microorganism.

15. The kit of claim 14, wherein said microspheres are streptavidin-coated microspheres.

16. The kit of claim 14, wherein said target microorganism is a bacterium, fungus, virus, or protozoan.

17. The kit of claim 14 wherein said microorganism is a pathogenic bacterium.

18. The kit of claim 17, wherein said bacterium is selected from the group consisting of Listeria, Escherichia, Salmonella, Campylobacter, Staphylococcus, and Streptococcus.

19. The kit of claim 14, wherein said bacterium is Listeria monocytogenes.

20. The kit of claim 14, wherein said primer pair is 5′-biotin-ATC ATC GAC GGC AAC CTC GGA GAC-3′ and 5′-biotin-CAC CAT TCC CM GCT AAA CCA GTG C-3′ and the target microorganism is Listeria monocytogenes.
